(19) United States
(12) Patent Application Publication
(10) Pub. No.: US 2021/0085374 A1
(43) Pub. Date: Mar. 25, 2021

(54) SYSTEMS AND METHODS FOR A PEDICLE SCREW ASSEMBLY

(71) Applicant: Dignity Health, San Francisco, CA (US)

(72) Inventor: Michael Austin Bohl, San Francisco, CA (US)

(21) Appl. No.: 17/115,632

(22) Filed: Dec. 8, 2020

Related U.S. Application Data
(62) Division of application No. 16/095,629, filed on Oct. 22, 2018.
(60) Provisional application No. 62/324,972, filed on Apr. 20, 2016.

(51) Int. Cl. A61B 17/70; A61B 17/86

(52) U.S. Cl. CPC A61B 17/7035; A61B 2017/8655; A61B 17/8625; A61B 17/864

(57) ABSTRACT

Various embodiments of a pedicle screw assembly having a pedicle screw defining an axial channel in communication with a pair of lateral channels configures to receive a bone anchor therein that extends outwardly from the pair of lateral channels in a post-deployment position for anchoring the pedicle screw to bone tissue are disclosed.

### CROSS-REFERENCE TO RELATED APPLICATIONS

This application is a divisional patent application of U.S. patent application Ser. No. 16/095,629 filed Oct. 22, 2018, which is a 371 national patent application of PCT International Application No. PCT/US2017/028683 filed Apr. 20, 2017, which claims priority under 35 U.S.C. § 119 to U.S. Provisional Patent Application No. 62/324,972 filed Apr. 20, 2016 entitled “SYSTEMS AND METHODS FOR A PEDICLE SCREW ASSEMBLY, all of which are incorporated by reference in their entireties herein.

### FIELD

The present disclosure generally relates to surgical devices and in particular to systems and methods for pedicle screw assemblies.

### BACKGROUND

Mechanical instability of the spine can result from many causes, including degenerative disease, trauma, infection, spinal deformity, or neoplastic processes. Left untreated, such spinal instability can result in pain, neurological compromise, and immobility. Spinal instability is treated surgically via fixation and fusion of the unstable spinal levels. Fixation of the spine is accomplished posteriorly through the insertion of screws through the vertebral pedicles, which are then segmentally connected by metal rods.

For patients undergoing spinal fixation and fusion surgery, pedicle screws are considered a standard of care. One of the most common complications associated with pedicle screw fixation, especially in long fixation constructs, is screw failure and pull-out with resultant pseudarthrosis or adjacent segment disease. Existing strategies for increasing the axial resistance required to pull out a pedicle screw include increased screw sizes, alternative core shapes, alternative thread shapes, and bone-cement augmentation. Each of these strategies comes with significant limitations, and none have proven to be a clinically reliable solution to the problem of pedicle screw failure.

It is with these observations in mind, among others, that various aspects of the present disclosure were conceived and developed.

### BRIEF DESCRIPTION OF THE DRAWINGS

FIG. 1 is a perspective view of a first embodiment of a pedicle screw assembly having a pedicle screw and a bone anchor, according to one aspect of the present disclosure;

FIG. 2 is an exploded view of the pedicle screw assembly of FIG. 1 depicting the pedicle screw and the bone anchor, according to one aspect of the present disclosure;

FIG. 3 is a cross-sectional view of the pedicle screw taken along line 3-3 of FIG. 2, according to one aspect of the present disclosure;

FIG. 4 is a cross-sectional view of the pedicle screw of FIG. 3 showing the bone anchor disposed partially within a channel of the pedicle screw, according to one aspect of the present disclosure;

FIG. 5 is a cross-sectional view of the pedicle screw of FIG. 3 showing the bone anchor disposed fully within the channel of the pedicle screw and deployed through an opening formed at the distal end of the pedicle screw, according to one aspect of the present disclosure;

FIG. 6 is an anatomical perspective view of the pedicle screw partially engaged inside a pedicle vertebra with the bone anchor in a pre-deployment position, according to one aspect of the present disclosure;

FIG. 7 is an anatomical perspective view of the pedicle screw partially engaged inside a pedicle vertebra with the anchor in a post-deployment position, according to one aspect of the present disclosure;

FIG. 8 is a perspective view of a second embodiment of a pedicle screw assembly showing a pedicle screw having a pair of bone anchors deployed from a pedicle screw by a driveshaft, according to one aspect of the present disclosure;

FIG. 9 is a perspective view of the pedicle screw of FIG. 8, according to one aspect of the present disclosure;

FIG. 10 is a cross-sectional view of the pedicle screw taken along line 10-10 of FIG. 9, according to one aspect of the present disclosure;

FIG. 11 is an exploded view of the pedicle screw assembly of FIG. 8 showing the pedicle screw, driveshaft and pair of bone anchors, according to aspects of the present disclosure;

FIG. 12 is a perspective view of the driveshaft, according to one aspect of the present disclosure

FIG. 13 is an end view of the driveshaft showing the spline, according to one aspect of the present disclosure;

FIG. 14 is a side view of the driveshaft, according to one aspect of the present disclosure;

FIG. 15 is a perspective view of one of the bone anchors, according to one aspect of the present disclosure;

FIG. 16 is a side view of the bone anchor of FIG. 15 showing a gear teeth arrangement, according to one aspect of the present disclosure;

FIG. 17 is an opposite side view of the bone anchor of FIG. 16, according to one aspect of the present disclosure;

FIG. 18 is a cross-sectional view of the pedicle screw assembly showing the pair of bone anchors in a pre-deployment position, according to one aspect of the present disclosure;

FIG. 19 is a cross-sectional view of the pedicle screw assembly showing the pair of bone anchors in a post-deployment position, according to one aspect of the present disclosure;

FIG. 20 is a top view of the pedicle screw assembly of FIG. 19 showing the pair of bone anchors in the post-deployment position, according to one aspect of the present disclosure;

FIG. 21A is a cross-sectional view of the pedicle screw assembly showing the pair of bone anchors in the pre-deployment position shown in FIG. 18; and FIG. 21B is a cross-sectional view of the pedicle screw assembly showing the pair of bone anchors in the post-deployment position when the driveshaft is actuated, according to one aspect of the present disclosure;

FIG. 22 is an anatomical perspective view of the pedicle screw partially engaged inside a pedicle vertebra with the pair of anchors in the pre-deployment position, according to one aspect of the present disclosure;

FIG. 23 is an anatomical perspective view of the pedicle screw partially engaged inside a pedicle vertebra with the pair of bone anchors in the post-deployment position, according to one aspect of the present disclosure;

FIG. 24 is a perspective view of a third embodiment of the pedicle screw assembly showing a pedicle screw and bone anchor, according to one aspect of the present disclosure;

FIG. 25 is an exploded view of the pedicle screw assembly of FIG. 24 showing the pedicle screw and anchor, according to one aspect of the present disclosure;

FIG. 26 is a cross-sectional view of the pedicle screw taken along line 26-26 of FIG. 25, according to one aspect of the present disclosure;

FIG. 27 is a cross-sectional view of the pedicle screw showing the bone anchor in the pre-deployment position, according to one aspect of the present disclosure;

FIG. 28 is a cross-sectional view of the pedicle screw showing the bone anchor in the post-deployment position, according to one aspect of the present disclosure;

FIG. 29 is an anatomical perspective view of the pedicle screw partially engaged inside the pedicle vertebra with the anchor shown in the pre-deployment position, according to one aspect of the present disclosure;

FIG. 30 is an anatomical perspective view of the pedicle screw partially engaged inside the pedicle vertebra with the bone anchor shown in the post-deployment position, according to one aspect of the present disclosure;

FIG. 31 is a perspective view of a fourth embodiment of the pedicle screw assembly showing a pedicle screw and bone anchor, according to one aspect of the present disclosure;

FIG. 32 is an exploded view of the pedicle screw assembly of FIG. 31 showing the pedicle screw and bone anchor, according to one aspect of the present disclosure;

FIG. 33 is a cross-sectional view of the pedicle screw assembly taken along line 33-33 of FIG. 32, according to one aspect of the present disclosure;

FIG. 34 is a cross-sectional view of the pedicle screw assembly showing the bone anchor in a pre-deployment position, according to one aspect of the present disclosure;

FIG. 35 is a cross-sectional view of the pedicle screw showing the bone anchor in the post-deployment position, according to one aspect of the present disclosure;

FIG. 36 is an anatomical perspective view of the pedicle screw partially engaged inside the pedicle vertebra with the bone anchor shown in the pre-deployment position, according to one aspect of the present disclosure; and

FIG. 37 is an anatomical perspective view of the pedicle screw partially engaged inside the pedicle vertebra with the bone anchor shown in the post-deployment position, according to one aspect of the present disclosure.

Corresponding reference characters indicate corresponding elements among the view of the drawings. The headings used in the figures do not limit the scope of the claims.

### DETAILED DESCRIPTION

Various embodiments of a pedicle screw assembly having one or more bone anchors that are actuated for securing a pedicle screw within a pedicle of a vertebra are disclosed. In some embodiments, the pedicle screw assembly includes a pedicle screw having external threads configured to allow the pedicle screw to be partially engaged within a pedicle vertebra. In addition, various embodiments of the pedicle screw may include a pair of opposite distal openings configured to allow passage of a bone anchor through an axial channel of the pedicle screw. This allows the bone anchor to extend outwardly from the pedicle screw when the bone anchor is in a post-deployment position to further engage the pedicle screw to the pedicle vertebra.

In some embodiments, the bone anchor includes an elongated body having a bendable portion that allows a distal portion of the bone anchor to be oriented at an angle relative to the longitudinal axis of the pedicle screw when extending outwardly from one of the distal openings. In some embodiments, the bone anchor includes an elongated body having a distal portion that defines a first lateral extension and a second lateral extension that are biased to be oriented at an acute angle relative to the longitudinal axis of the pedicle screw when the bone anchor extends outwardly from the distal openings of the pedicle screw in a post-deployment position. In some embodiments, the bone anchor includes an elongated body having a distal portion that defines a first lateral extension and a second lateral extension that are biased to be oriented at an obtuse angle relative to the longitudinal axis of the pedicle screw when the bone anchor extends outwardly from the distal openings of the pedicle screw in the post-deployment position. In some embodiments, the bone anchor may be a first bone anchor and a second bone anchor each having gear teeth configured to engage a spline portion of a driveshaft such that rotation of the driveshaft causes the first and second bone anchors to extend laterally outward from the distal openings of the pedicle screw in a post-deployment position.

In some embodiments, the pair of opposite distal openings of the pedicle screw may communicate with respective first and second lateral channels that are oriented at an obtuse angle relative to the longitudinal axis of the pedicle screw. In some embodiments, the pair of opposite distal openings of the pedicle screw may communicate with respective first and second lateral channels that are oriented at an acute angle relative to the longitudinal axis of the pedicle screw. In some embodiments, the pair of opposite distal openings of the pedicle screw may communicate with respective first and second lateral channels that are oriented at a perpendicular angle relative to the longitudinal axis of the pedicle screw.

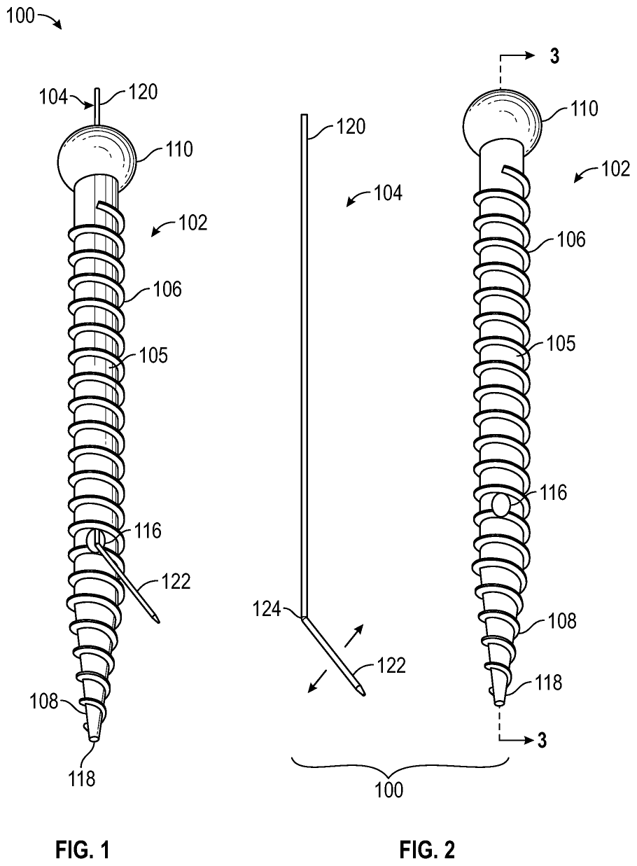

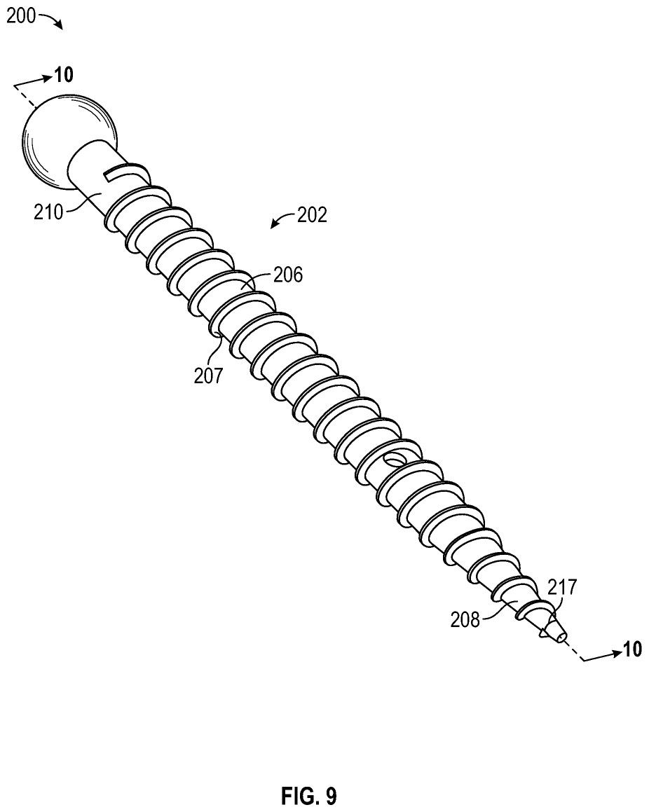

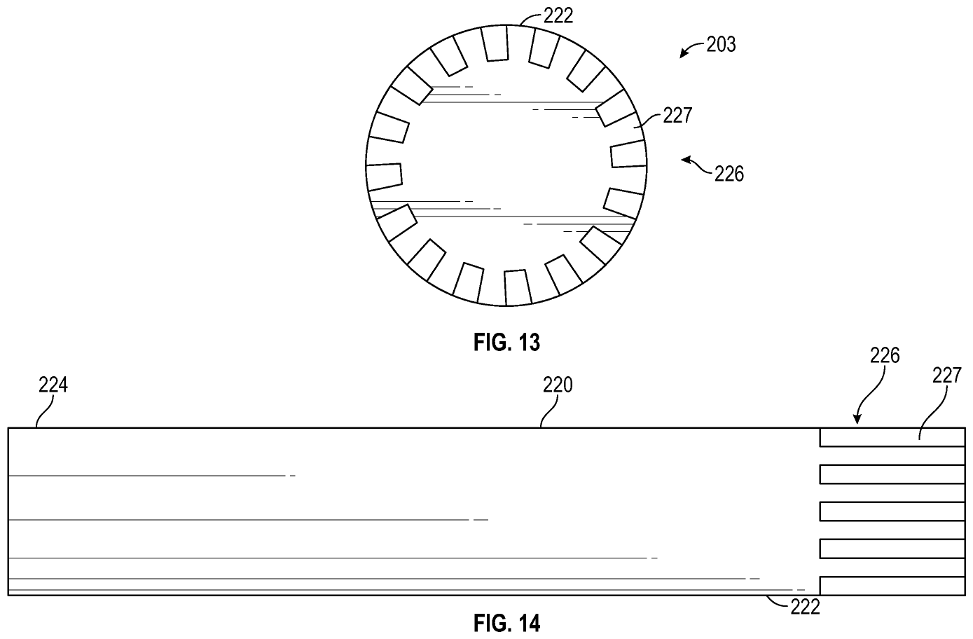

In one method of engaging a pedicle screw to a bone portion, a pedicle screw is inserted into a pedicle of the vertebra and a bone anchor is inserted through the pedicle screw such that the distal portion of the bone anchor extends outwardly from the pedicle screw in a post-deployment position to further engage the pedicle screw to the pedicle vertebra. In another method of engaging a pedicle screw to a bone portion, a pedicle screw is inserted into a pedicle of the vertebra and a pair of bone anchors disposed within one or more respective lateral channels in a pre-deployment position are caused to extend outwardly to a post-deployment position from the pedicle screw upon actuation of a driveshaft that is operatively engaged to each bone anchor to further engage the pedicle screw to the pedicle vertebra. Referring to the drawings, embodiments of a pedicle screw assembly are illustrated and generally indicated as 100, 200, 300 and 400 in FIGS. 1-37.

Referring to FIGS. 1-7, a first embodiment of a pedicle screw assembly, designated 100, is illustrated. As shown in FIGS. 1-3, the pedicle screw assembly 100 includes a pedicle screw 102 that is configured to be coupled to a bone anchor 104 for engaging a portion of tissue, such as the bone tissue of the vertebra. In some embodiments, the pedicle screw 102 defines an elongated body 105 forming a distal portion 108 defining a conical tip 118 at the free end thereof and a proximal portion 110 that defines a proximal opening 114 in communication with an axial channel 112 formed along the longitudinal axis of the pedicle screw 102. In some embodiments, the elongated body 105 defines a threaded portion 106 that extends substantially along the length of the elongated body 105 configured to permit the pedicle screw 102 to engage and retained within the bone tissue.

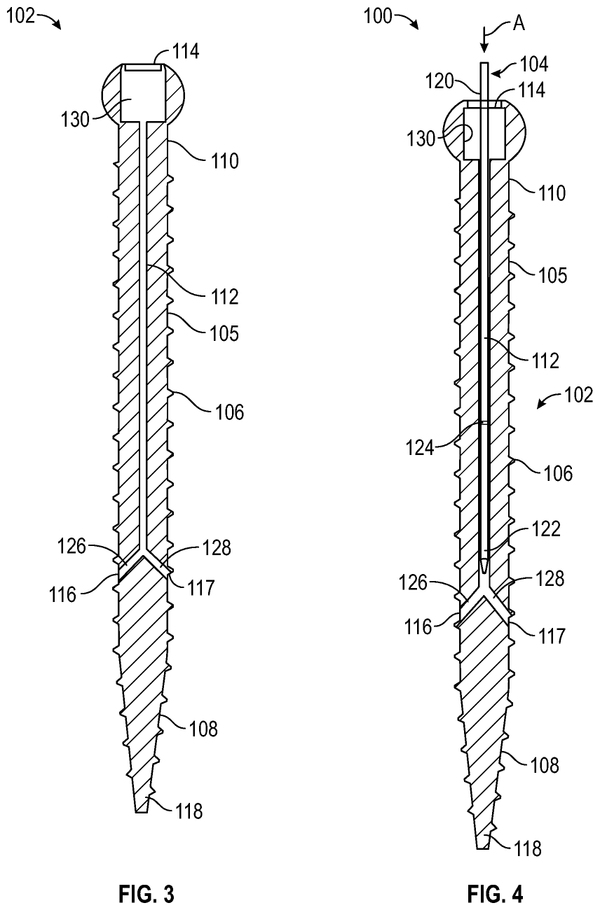

Referring to FIG. 3, proximal opening 114 communicates with the one end of the axial channel 112 through a cavity 130 formed proximate the proximal opening 114, while the opposite end of the axial channel 112 communicates with a pair of first and second lateral channels 116 and 117 that extend at an obtuse angle (downward angle) relative to the axial channel 112. As shown, the first lateral channel 126 communicates with a first lateral opening 116 and the second lateral channel 128 communicates with a second lateral opening 117 which are both formed along the distal portion 108 of the pedicle screw 102.

Referring back to FIG. 2, in some embodiments the bone anchor 104 defines an elongated proximal portion 120 and a distal portion 122 configured to be received through the axial channel 112 and one of either the first or second lateral channels 126 and 128 such that the distal portion 122 of the bone anchor 104 extends outwardly from either the first or second lateral openings 116 and 117 in a post-deployment position. In some embodiments, the bone anchor 104 defines a bendable portion 124 formed between the proximal portion 120 and distal portion 122 of the bone anchor 104 that allows the distal portion 122 of the bone anchor 104 to bend or hinge relative to the proximal portion 120 as the bone anchor 104 extends through either the first or second lateral openings 116 and 117.

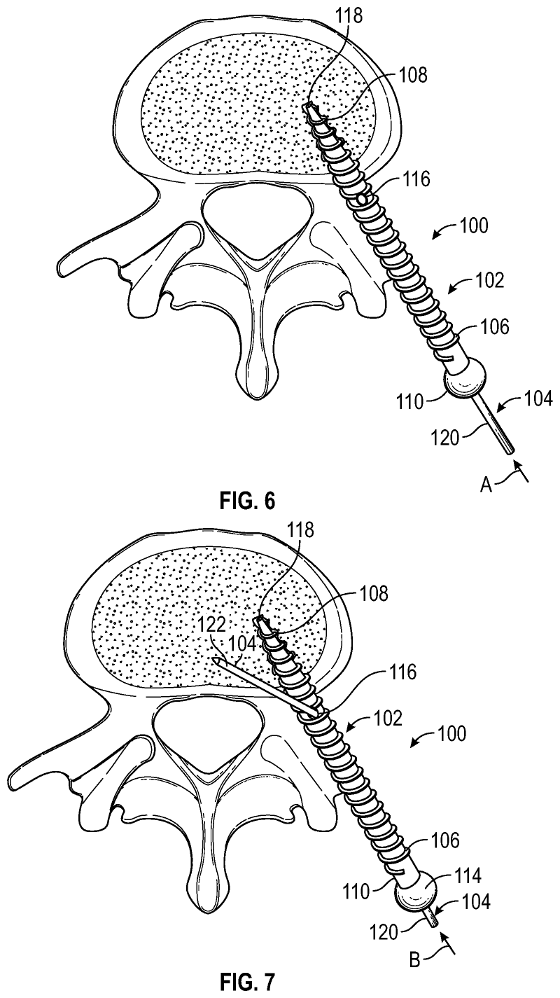

Referring to FIGS. 4-7, one method of implanting the pedicle screw assembly 100 will be discussed. As shown in FIG. 6, the pedicle screw 102 is first engaged into the bone tissue, for example a pedicle of the vertebra, such that the proximal portion 110 of the pedicle screw 102 extends from the bone tissue. Once the pedicle screw 102 is implanted into the bone tissue, the bone anchor 104 is inserted into the proximal opening 114 of the pedicle screw 102 along direction A until disposed within the axial channel 112 proximate the first and second lateral channels 126 and 128 in a pre-deployment position as illustrated in FIG. 4.

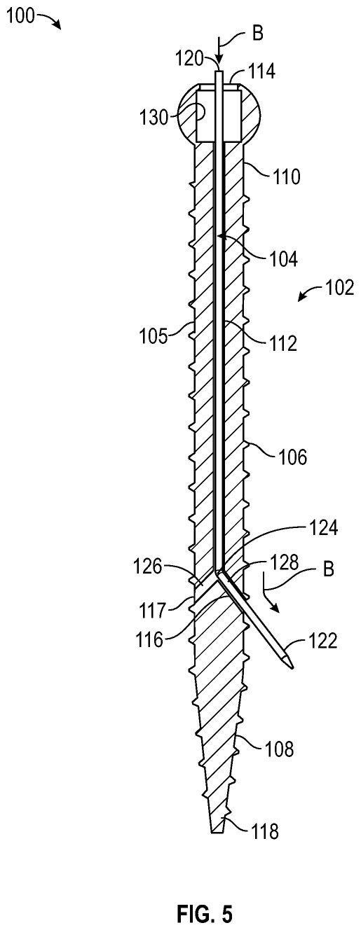

Once in the pre-deployment position, in some embodiments the user may then further insert the bone anchor 104 in direction B until the distal portion 122 of the bone anchor 104 travels through either the first or second lateral channels 126 and 128 until extending outwardly from either the first or second lateral openings 116 and 117 in a post-deployment position as illustrated in FIG. 5. In this post-deployment position, the distal portion 122 of the bone anchor is engaged to the bone tissue as shown in FIG. 7.

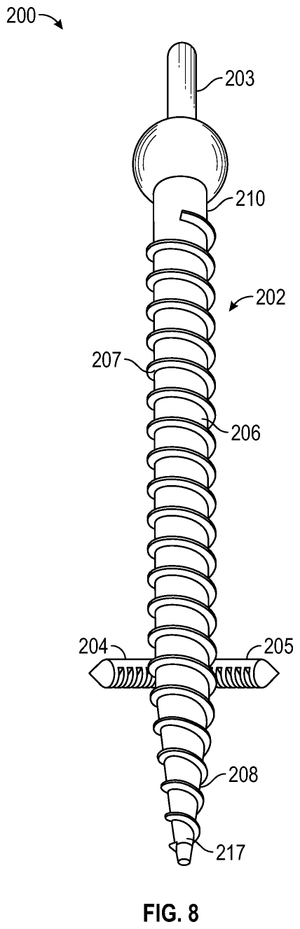

Referring to FIGS. 8-23, a second embodiment of the pedicle screw assembly, designated 200, is illustrated. As shown in FIGS. 8-11, the pedicle screw assembly 200 includes a pedicle screw 202 that is configured to be coupled to a driveshaft 203 which is in operative engagement with a pair of first and second bone anchors 204 and 205 for anchoring the pedicle screw 202 into bone tissue, such as a pedicle vertebra. In some embodiments, the pedicle screw 202 defines an elongated body 206 forming a distal portion 208 defining a conical tip 217 at the free end thereof and a proximal portion 210 that defines a proximal opening 216 in communication with an axial channel 212 formed along the longitudinal axis of the pedicle screw 202. In some embodiments, the elongated body 206 defines a threaded portion 207 that extends substantially along the length of the elongated body 206 configured to permit the pedicle screw 202 to engage and be retained within the bone tissue.

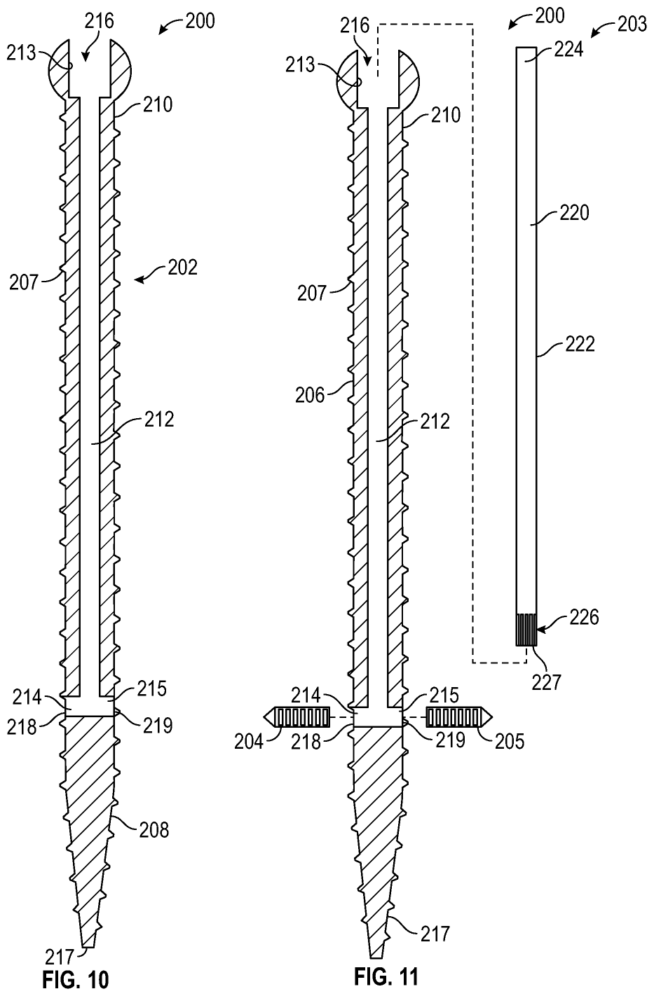

Referring to FIGS. 10 and 11, proximal opening 216 communicates with the one end of the axial channel 212 through a cavity 213 formed proximate the proximal opening 216, while the opposite end of the axial channel 212 communicates with a pair of first and second lateral channels 214 and 215 that extend at a substantially perpendicular angle relative to the longitudinal axis of the axial channel 212. As shown, the first lateral channel 214 communicates with a first lateral opening 218 and the second lateral channel 215 communicates with a second lateral opening 219 which are both formed generally adjacent to the distal portion 208 of the pedicle screw 202.

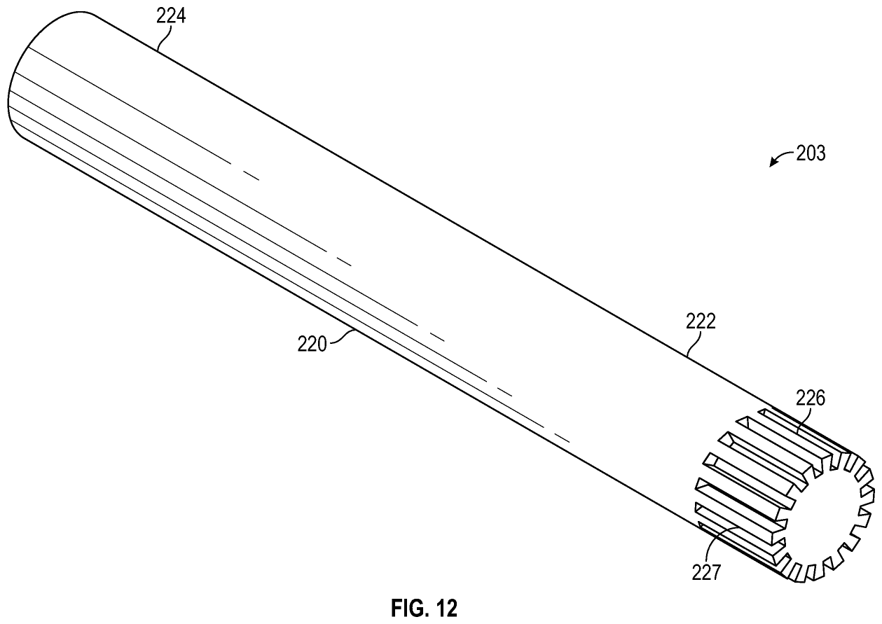

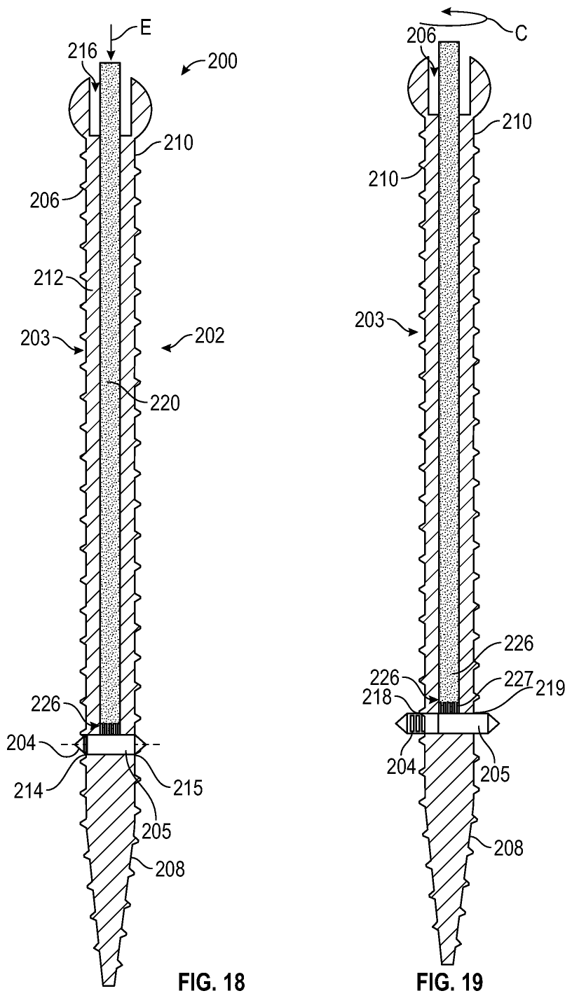

Referring specifically to FIGS. 12-14, 18 and 19, the driveshaft 203 is operative to actuate the first and second bone anchors 204 and 205 from a pre-deployment position (FIG. 18) with the first and second bone anchors 204 and 205 fully disposed within the first and second lateral channels 214 and 215, respectively, and a post-deployment position (FIG. 19) with the first and second bone anchors 204 and 205 extending outwardly from the first and second channels 214 and 215, respectively. In operation, rotation of the driveshaft 203 causes the first and second bone anchors 204 and 205 to extend laterally to the post-deployment position from the first and second lateral openings 218 and 219, respectively, as shall be discussed in greater detail below.

Referring to FIGS. 12-14, in some embodiments the driveshaft 203 defines an elongated body 220 defining a distal portion 222 and a proximal portion 224. As shown, the distal portion 222 of the drive shaft 203 defines a spline 226 that forms a plurality of axially extending peripheral slots 227 formed along the periphery of the spline 226. The spline 226 is configured to operatively engage at least one of the first and second bone anchors 204 and 205 as discussed below.

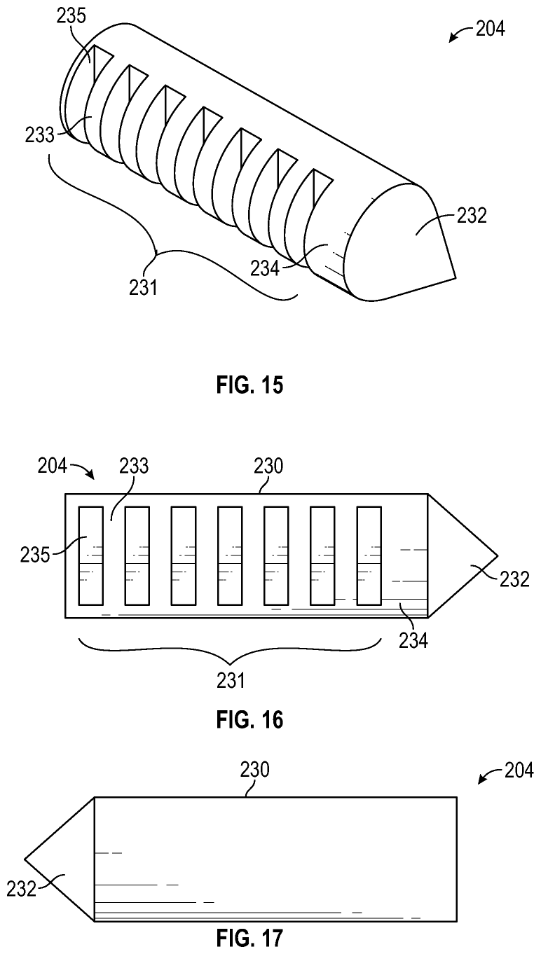

In some embodiments, the first and second bone anchors 204 and 205 have identical shapes and are configured to be disposed within the first and second lateral channels 214 and 215, respectively, of the pedicle screw 202 when in the pre-deployment position shown in FIG. 18. By way of example, the first bone anchor 204, which is identical to the second bone anchor 205, will be described in reference to FIGS. 15-17 with the description being applicable to both first and second bone anchors 204 and 205. In some embodiments, each bone anchor 204 and 205 has an anchor body 230 defining a pointed end 232 configured to penetrate bone tissue when the first and second bone anchors 204 and 205 are in the post-deployment position as shown in FIG. 8. In addition, the anchor body 230 has a lateral side 234 forming a gear teeth arrangement 231 configured to engage the spline 226 of the driveshaft 203. In some embodiments, the gear teeth arrangement 231 defines a plurality of ridges 233 and slots 235 in which each ridge 233 is formed between a respective pair of slots 235 along a length of the gear teeth arrangement 231. In one method of engagement between the first and second bone anchors 204 and 205 and the driveshaft 203, a respective portion of the spline 226 for the driveshaft 203 engages the respective gear teeth arrangement 233 of the first and second bone anchors 204 and 205 such that rotation of the driveshaft 203 advances the first and second bone anchors 204 and 205 outwardly from the pedicle screw 202.

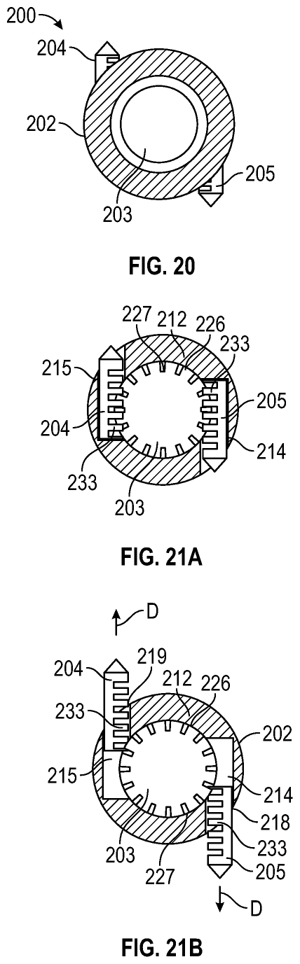

As noted above, the driveshaft 203 is configured to be disposed within the axial channel 212 such that the spline 226 operatively engages the first and second bone anchors 204 and 205 disposed within the first and second lateral channels 214 and 215, respectively, when the pedicle screw assembly 100 is in the pre-deployment position as shown in FIG. 18. In the pre-deployment position shown in the cross-sectional views of FIGS. 18 and 21A, the first and second anchors 204 and 205 are disposed within the first and second lateral channels 214 and 215 such that the gear teeth arrangement 233 of the first and second bone anchors 204 and 205 are operatively engaged to the spline 226 of the driveshaft 203. In the post-deployment position shown in the cross-sectional views of FIGS. 19 and 21B, rotation of the driveshaft 203 in a counter-clockwise direction causes the spline 226 to continuously engage respective gear teeth arrangement 233 such that the first and second bone anchors 204 and 205 are incrementally moved outwardly from the first and second lateral channels 214 and 215 as illustrated in FIG. 20. In other embodiments, the driveshaft 203 and the first and second bone anchors 204 and 205 may be operatively engaged such that rotation of the driveshaft 203 in the opposite clockwise direction also incrementally moves the first and second bone anchors 204 and 205 outwardly from the first and second lateral channels 214 and 215.

Referring back to FIG. 21A, in some embodiments the first and second lateral channels 214 and 215 are formed in an off-set arrangement on either side of the axial channel 212 and are in communication thereto. As such, the gear teeth arrangement 233 of the first and second bone anchors 204 and 205 are in operative engagement with the spline 226 when the driveshaft 203 is fully disposed within the axial channel 212.

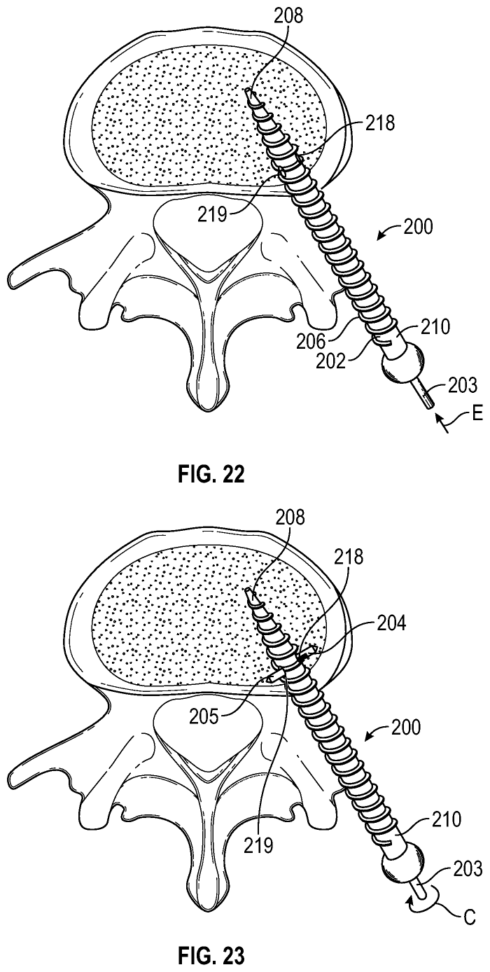

Referring to FIGS. 22 and 23, one method of implanting the pedicle screw assembly 200 will be discussed. As shown in FIG. 22, the pedicle screw 202 is first engaged into the bone tissue, such as a pedicle vertebra, such that the proximal portion 210 of the pedicle screw 102 extends from the bone tissue and the distal portion 208 of the pedicle screw 202 is partially or fully implanted inside the bone tissue. Once the pedicle screw 202 is implanted into the bone tissue, the driveshaft 203 is inserted into the axial channel 212 of the pedicle screw 202 along axial direction E until disposed within the axial channel 212 proximate the first and second lateral channels 214 and 215. This insertion of the driveshaft 203 into the pedicle screw 202 allows the driveshaft 203 to be in operative engagement with the first and second bone anchors 204 and 205 already disposed within the first and second lateral channels 214 and 215, respectively, thereby placing the pedicle screw assembly 200 is in a pre-deployment position.

Once in the pre-deployment position, the user may then rotate the driveshaft 203 in the counter-clockwise direction C until the first and second bone anchors 204 and 205 are fully extended into the bone tissue by the rotation of the driveshaft 203. As such, the pedicle screw assembly 200 assumes a post-deployment position as shown in FIG. 23.

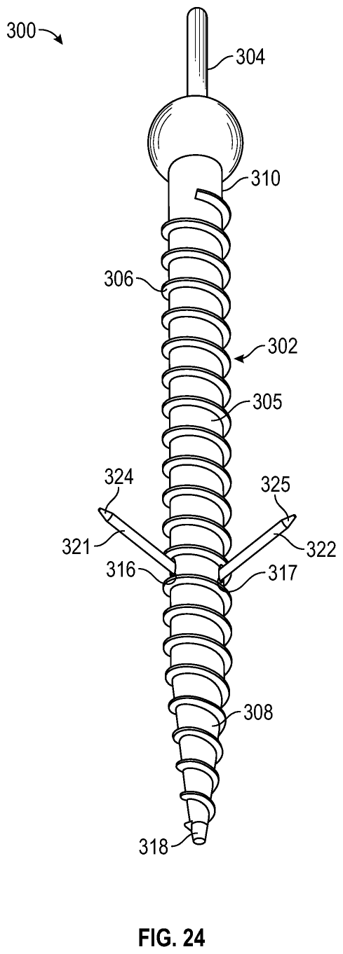

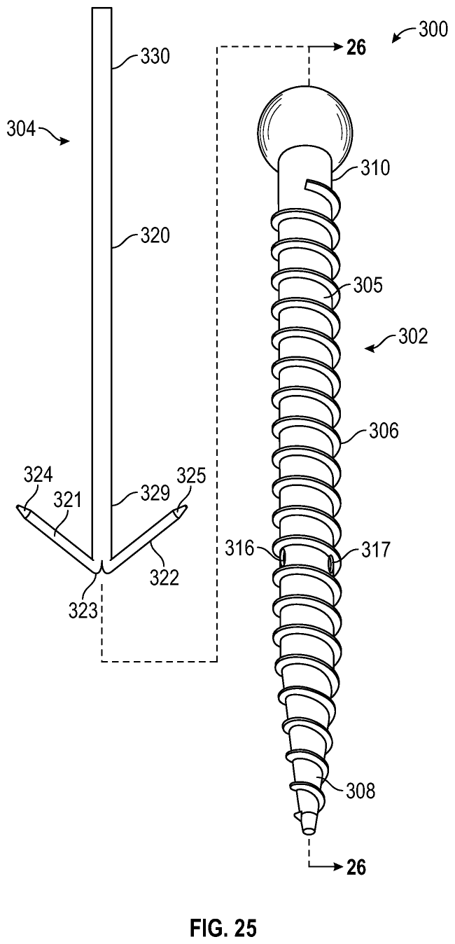

Referring to FIGS. 24-30, a third embodiment of a pedicle screw assembly, designated 300, is illustrated. As shown in FIGS. 24 and 25, the pedicle screw assembly 300 includes a pedicle screw 302 that is configured to be coupled to a bone anchor 304 for engaging a portion of bone, such as a pedicle vertebra. In some embodiments, the pedicle screw 302 defines an elongated body 305 forming a distal portion 308 defining a conical tip 318 at the free end thereof and a proximal portion 310 that defines a proximal opening 314 in communication with an axial channel 312 formed along the longitudinal axis of the pedicle screw 302. In some embodiments, the elongated body 305 defines a threaded portion 306 that extends substantially along the length of the elongated body 305 configured to permit the pedicle screw 302 to engage and be retained within the bone tissue.

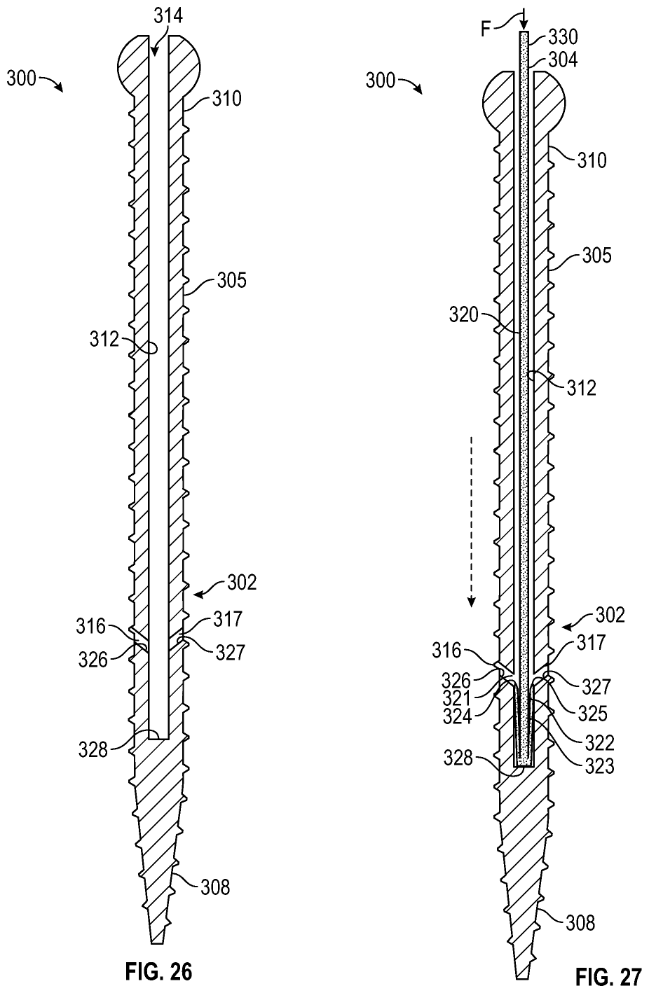

Referring to FIG. 26, the proximal opening 314 communicates with one end of the axial channel 312, while the opposite end of the axial channel 312 communicates with a pair of first and second lateral channels 326 and 327 that extend at an acute (upward) angle relative to longitudinal axis of the axial channel 312. As shown, the first lateral channel 326 communicates with a first lateral opening 316 and the second lateral channel 327 communicates with a second lateral opening 317 which are both formed along the distal portion 308 of the pedicle screw 302.

Referring back to FIGS. 24 and 25, in some embodiments the bone anchor 304 defines an elongated body 320 forming an elongated proximal portion 330 and a distal portion 329 configured to be received through the axial channel 312 of the pedicle screw 302. The bone anchor 304 further defines an apex 323 at the distal portion 329 with a first lateral extension 321 and a second lateral extension 322 extending from the apex 323 and the distal portion 329 of the bone anchor 304. In some embodiments, the first lateral extension 321 of the bone anchor 304 includes a first pointed end 324, and the second lateral extension 322 of the bone anchor 304 includes a second pointed end 325. The bone anchor 304 is depicted in FIG. 25 in an unbiased state with the first lateral extension 321 and the second lateral extension 322 extending laterally away from the elongated body 320 of the bone anchor 304.

In a pre-deployment position, the bone anchor 304 is inserted into and disposed within the axial channel 312 in direction F (FIG. 27) and assumes a biased state as the first lateral extension 321 and the second lateral extension 322 are forced together toward the elongated proximal portion 330 due to the small width of the axial channel 312. Specifically, the first lateral extension 321 and the second lateral extension 322 become temporarily biased together or otherwise bent inwardly toward each other within the axial channel 312 such that the first and second pointed ends 324 and 325 are positioned generally adjacent to the first and second lateral channels 326 and 327 as illustrated in FIG. 27 In other words, manipulating the first lateral extension 321 and the second lateral extension 322 into the pre-deployment position described above temporarily configures the bone anchor 304 in a substantially linear shape which allows the bone anchor 304 to be slidably received within the axial channel 312 of the pedicle screw 302. The bone anchor may be urged all the way through the axial channel 312 such that the apex 323 of the bone anchor 304 makes contact with or at least substantially reaches a terminal ending 328 of the axial channel 312 of the pedicle screw 302.

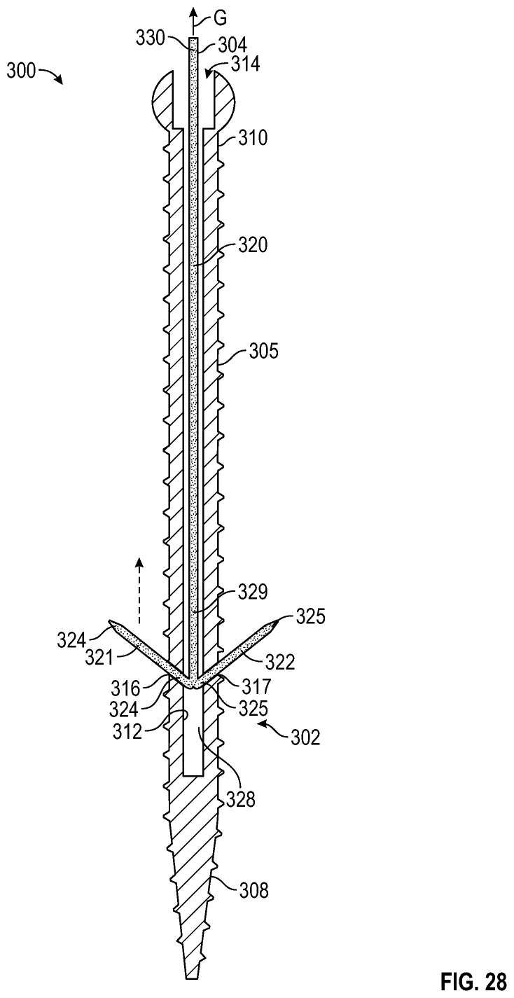

Referring to FIGS. 27-28, the bone anchor 304 may be manipulated from a pre-deployment position to a post-deployment position with the first lateral extension 321 and the second lateral extension 322 oriented away from the elongated body 320 and extending through the first lateral opening 316 and the second lateral opening 317, respectively. In this post-deployment position, the first lateral extension 321 and the second lateral extension 322 generally return to an unbiased state. Specifically, to assume the post-deployment position, the bone anchor 304 is pulled in a direction G (FIG. 28) opposite the direction F to orient the bone anchor 304 such that the first pointed end 324 and the second pointed end 325 positioned below the first lateral opening 316 and the second lateral opening 317 respectively may then spring through the first lateral channel 326 and the first lateral opening 316, and the second lateral extension 322 may spring through the second lateral channel 327 and the second lateral opening 317 to assume the post-deployment position shown in FIG. 28.

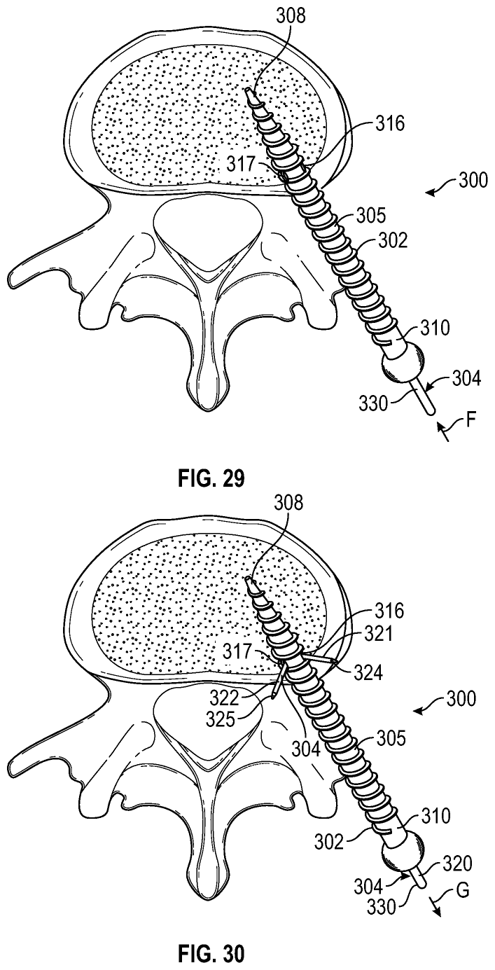

Referring to FIGS. 29-30, one method of implanting the pedicle screw assembly 300 will be discussed. As shown in FIG. 29, the pedicle screw 302 is first engaged into a bone tissue, such as a pedicle vertebra, such that the proximal portion 310 of the pedicle screw 302 extends from the bone tissue. Once the pedicle screw 302 is implanted into the bone tissue, the bone anchor 304 is inserted through the proximal opening 314 and into the axial channel 312 of the pedicle screw 302 along the direction F until disposed within the axial channel 312 proximate the first and second lateral channels 326 and 327. The bone anchor 304 may then be pulled back slightly in the direction G opposite the direction F to bring the bone anchor 304 into the post-deployment position of FIG. 30. In the post deployment position, the first lateral extension 321 extends through the first lateral opening 316 with the first pointed end 324 extending into the bone tissue, and the second lateral extension 322 extends through the second lateral opening 317 with the second pointed end 325 extending into the bone tissue.

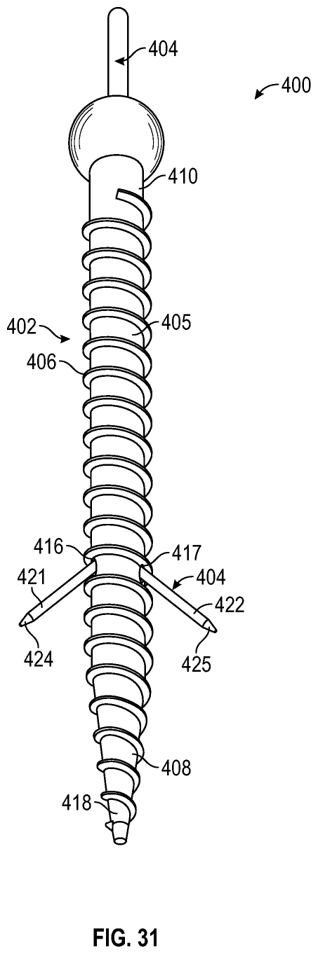

Referring to FIGS. 31-37, a fourth embodiment of the pedicle screw assembly, designated 400 is illustrated. As shown in FIGS. 31-33, the pedicle screw assembly 400 includes a pedicle screw 402 that is configured to be coupled to a bone anchor 404 for engaging a portion of bone tissue, such as a pedicle vertebra. In some embodiments, the pedicle screw 402 defines an elongated body 405 forming a distal portion 408 defining a conical tip 418 at the free end thereof and a proximal portion 410 that defines a proximal opening 414 in communication with an axial channel 412 formed along the longitudinal axis of the pedicle screw 402. In some embodiments, the elongated body 405 defines a threaded portion 406 substantially along the length of the elongated body 405 to permit the pedicle screw 402 to be screwed into and retained within the bone tissue.

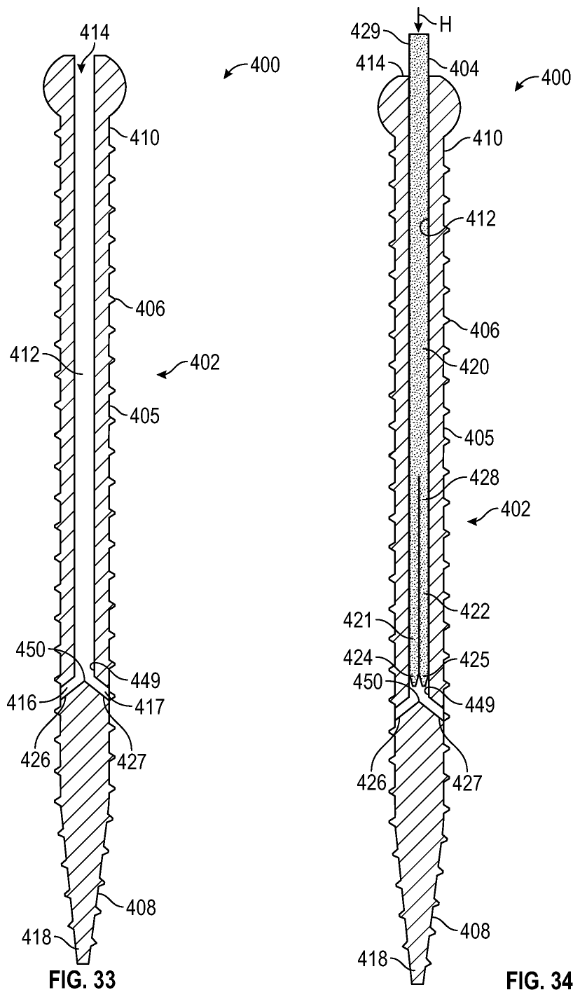

Referring to FIG. 33, the proximal opening 414 communicates with one end of the axial channel 412, while the opposite end of the axial channel 412 communicates with a pair of first and second lateral channels 426 and 427 that extend at an obtuse angle (downward angle) relative to the axial channel 412. As shown, the first lateral channel 426 communicates with a first lateral opening 416 and the second lateral channel 427 communicates with a second lateral opening 417 which are both formed along the distal portion 408 of the pedicle screw 402. An apex 450 is formed at a terminal end 449 of the axial channel 412 and may be defined between the first and second lateral channels 426 and 427. In some embodiments, the apex 450 is triangle-shaped to facilitate the guidance of the first lateral extension 421 through the first lateral channel 426, and the second lateral extension 422 through the second lateral channel 427, as described herein.

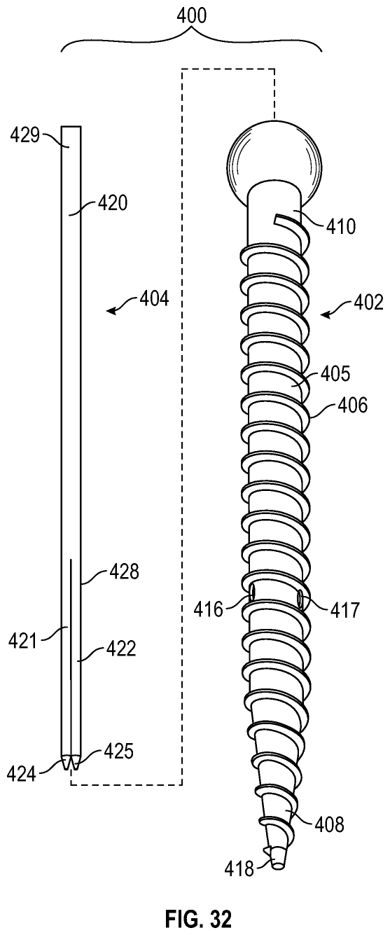

Referring back to FIG. 32, in some embodiments the bone anchor 404 defines an elongated proximal portion 420 and a distal portion 428. The bone anchor 404 further defines the first lateral extension 421 and the second lateral extension 422 defined at the distal portion 428. In some embodiments, the first lateral extension 421 may include a first pointed end 424 and the second lateral extension 422 may include a second pointed end 425. In a pre-deployment position shown in FIG. 32, the first lateral extension 421 and the second lateral extension 422 may be arranged in a substantially parallel arrangement. In some embodiments, a slight gap, or space, may be defined between the first lateral extension 421 and the second lateral extension 422. In some embodiments, a thin, breakable connection material, such as a plastic or thin sheet of metal, may be formed between the first lateral extension 421 and the second lateral extension 422 to temporarily maintain the first lateral extension 421 and the second lateral extension 422 in the pre-deployment position. In some embodiments, the bone anchor 404 defines a bendable portion at the base of the first lateral extension 421 and the second lateral extension 422 between the proximal portion 420 and distal portion 428 of the bone anchor 404 that allows the first lateral extension 421 and the second lateral extension 422 of the bone anchor 104 to bend or hinge relative to the proximal portion 420.

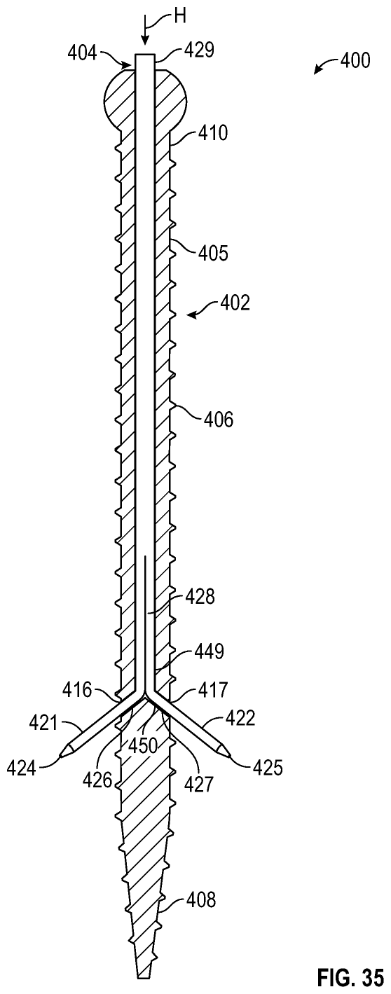

In a post-deployment position, the first lateral extension 421 and the second lateral extension 422 may be received through the axial channel 412 and the first and second lateral channels 426 and 427 respectively and extend outwardly from the first and second lateral openings 416 and 417 respectively. To render the pedicle screw assembly 400 in the post-deployment position, the bone anchor 404 may first be aligned over the pedicle screw 402 with the distal portion 428 oriented towards the proximal opening 414 of the pedicle screw 402. The bone anchor 404 may then be slidably engaged in a direction H through the proximal opening 414 and into the axial channel 412 of the pedicle screw 402 as shown in FIG. 34. Subsequently, the bone anchor 404 may continue to be moved in the direction H until the distal end 428 of the bone anchor 404 contacts the apex 450 formed at the terminal end of the axial channel 412 opposite the proximal opening 414. Continual insertion of the bone anchor 404 into the axial channel 412 drives the first pointed end 424 of the first lateral extension 421 and the second pointed end 425 of the second lateral extension 422 against the apex 450, thereby causing the first lateral extension 421 and the second lateral extension 422 to split away from each other. As such, the first lateral extension 421 passes through the first lateral channel 426 and extends outwardly from the first lateral opening 416, while the second lateral extension 422 passes through the second lateral channel 427 and extends outwardly from the second lateral opening 417 until the post-deployment position shown in FIG. 35 is achieved (i.e., an inverse “Y” configuration).

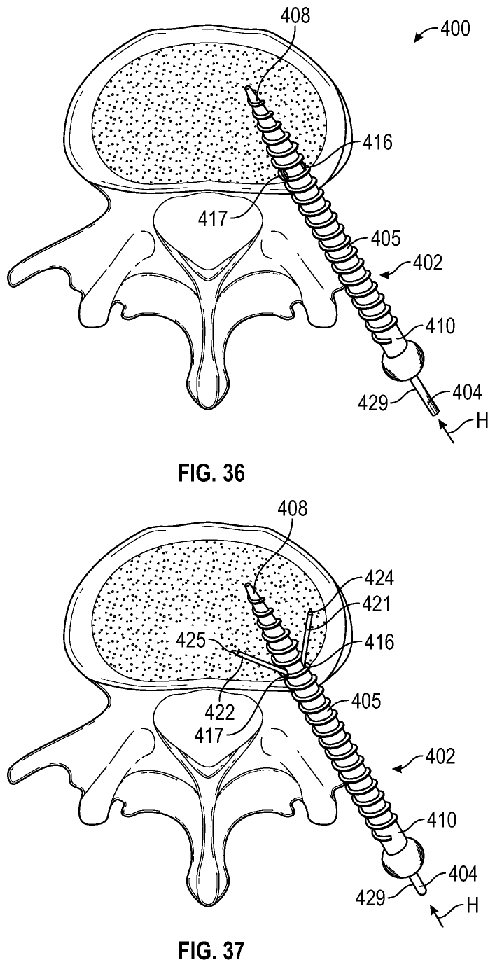

Referring to FIGS. 36-37, one method of implanting the pedicle screw assembly 400 will be discussed. As shown in FIG. 36, the pedicle screw 402 is first engaged into the bone tissue, such as a pedicle vertebra, such that the proximal portion 410 of the pedicle screw 402 extends from the bone tissue. Once the pedicle screw 402 is implanted into the bone tissue, the bone anchor 404 is inserted into the proximal opening 414 of the pedicle screw 402 until disposed within the axial channel 412 proximate the first and second lateral channels 426 and 427 in a pre-deployment position illustrated in FIG. 34.

Once in the pre-deployment position, in some embodiments the user may then further insert the bone anchor 404 until first lateral extension 421 and the second lateral extension of the bone anchor 404 travel through the first and second lateral channels 426 and 427 respectively until extending outwardly from the first and second lateral openings 416 and 417 respectively in a post-deployment position as illustrated in FIG. 35. In this post-deployment position, the distal portion 428 of the bone anchor 404 is engaged to the bone tissue as shown in FIG. 37.

In some embodiments, the pedicle screw assemblies 100, 200, 300 and 400 are configured to be affixed to the larger vertebrae of the lumbar spine, or the smaller vertebrae of the thoracic or cervical spine.

In some embodiments, the pedicle screw assemblies 100, 200, 300 and 400 may be made from a metal, such as titanium, or a metal-based alloy, such as titanium-based alloy. Alternatively, the pedicle screw assemblies 100, 200, 300 and 400 may be reinforced polymer material. In some embodiments, the material used to manufacture the pedicle screw assemblies 100, 200, 300 and 400 can have a high bioactivity and high flexibility, and a result, can improve ingrowth and mechanical fixation.

In some embodiments, the pedicle screws 102, 202, 302 and 402 may be engaged to a tulip structure (not shown) which is configured to interface with a longitudinal bar or a plate. In some embodiments, the tulip structure can be flexibly coupled to the pedicle screws 102, 202, 302, and 402 by way of a ball-joint or other type of flexible joint such that the pedicle screw assemblies 100, 200, 300, and 400 can account for any bending of the individual's spine while still exerting an axial force on the longitudinal bar, thereby stabilizing the spine of the individual.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A pedicle screw assembly, comprising:
a pedicle screw, comprising
a screw body, the screw body being elongated and defining a proximal portion and a distal portion,
an axial channel defined along a longitudinal axis of the screw body, the axial channel in communication with a proximal opening defined along the proximal portion of the screw body and defining a terminal end portion of the axial channel along the distal portion of the screw body,
a first lateral channel formed along the distal portion of the screw body and in communication with the axial channel,
a second lateral channel in communication the axial channel and defined opposite the first lateral channel, and
a first lateral opening in communication with the first lateral channel, the first lateral opening defined between an intersection of the first lateral channel and the axial channel and the proximal portion of the screw body; and
an anchor, comprising
an anchor body defining an anchor proximal portion and an anchor distal portion, and
a first lateral extension defined along the anchor distal portion,
wherein the axial channel is configured to receive the anchor body and the first lateral channel is configured to receive the first lateral extension of the anchor such that a first pointed end of the first lateral extension is oriented towards the proximal portion of the screw body.

2. The pedicle screw assembly of claim 1, wherein the first lateral extension includes a pointed end configured to drive the first lateral extension within bone tissue associated with a pedicle vertebra.

3. The pedicle screw assembly of claim 1, further comprising:
a bendable portion defined along the anchor body of the anchor, the first lateral extension being bendable relative to the anchor body at the bendable portion.

4. The pedicle screw assembly of claim 1, wherein the pedicle screw further comprises:
a second lateral opening in communication the second lateral channel defined along the screw body opposite the first lateral opening.

5. The pedicle screw assembly of claim 1, wherein the terminal end portion of the axial channel is defined proximate the first lateral channel and the second lateral channel.

6. The pedicle screw assembly of claim 1, wherein the anchor further comprises a second lateral extension defined along the anchor distal portion of the anchor.

7. The pedicle screw assembly of claim 6, wherein the first lateral extension and the second lateral extension of the bone anchor collectively form an apex such that the first and second lateral extensions are oriented adjacent the anchor proximal portion.

8. The pedicle screw assembly of claim 6, wherein the first lateral extension and the second lateral extension of the anchor are oriented in parallel relative to each other.

9. The pedicle screw assembly of claim 1, wherein the pedicle screw further defines a threaded portion configured for engagement within a pedicle vertebra.

10. A pedicle screw assembly, comprising:
a pedicle screw defining a distal portion and a proximal portion, the pedicle screw including an axial channel extending from the proximal portion to the distal portion and further defining a first lateral channel in communication with the axial channel, the first lateral channel defining a lateral opening defined between the distal portion and the proximal portion; and
an anchor including an anchor body defining an anchor proximal portion and an anchor distal portion, and a first lateral extension defined along the anchor distal portion,
wherein the axial channel is configured to receive the anchor body and the first lateral channel is configured to receive the first lateral extension of the anchor such that the first lateral extension is oriented towards the proximal portion of the pedicle screw.

11. The pedicle screw assembly of claim 10, wherein the first lateral extension of the anchor is biased away from the anchor body.

12. The pedicle screw assembly of claim 10, further comprising:
a second lateral extension extending from an apex of the anchor body, wherein, in an unbiased state, an acute angle is defined at intersection between the second lateral extension and the anchor body.

13. The pedicle screw assembly of claim 10, further comprising:
an apex of the axial channel defined between the proximal portion of the pedicle screw and a terminal end portion of the axial channel, the apex defined along an intersection of the first lateral channel and a second lateral channel with the axial channel.

14. A method, comprising:
providing a pedicle screw including an axial channel, a proximal opening in communication with the axial channel, and a lateral channel in communication with the axial channel;
providing a bone anchor defining an anchor proximal portion and an anchor distal portion, the bone anchor defining a lateral extension defined at the anchor distal portion;
orienting the anchor distal portion over the proximal opening of the pedicle screw;
disposing the bone anchor into the axial channel and pushing the bone anchor in a first direction; and
moving the lateral extension of the bone anchor through the lateral channel.

15. The method of claim 14, further comprising:
applying a force to the anchor proximal portion to drive the lateral extension through the lateral channel.

16. The method of claim 14, further comprising:
pulling the bone anchor through the axial channel to drive the lateral extension through the lateral channel.
